(19) United States
(12) Patent Application Publication
(10) Pub. No.: US 2026/0300742 A1
(43) Pub. Date: Oct. 1, 2026

(54) TRAINING AND USE OF A POSTURE INVARIANT BRAIN-COMPUTER INTERFACE

(71) Applicant: CARNEGIE MELLON UNIVERSITY, Pittsburgh, PA (US)

(72) Inventors: PATRICK MARINO, Pittsburgh, PA (US); AARON P. BATISTA, Pittsburgh, PA (US); BYRON M. YU, Pittsburgh, PA (US); STEVEN M. CHASE, Pittsburgh, PA (US); ASMA NAUSHAD MOTIWALA, Pittsburgh, PA (US); EMILY R. OBY, Pittsburgh, PA (US); ADAM L. SMOULDER, Pittsburgh, PA (US)

(73) Assignee: CARNEGIE MELLON UNIVERSITY, Pittsburgh, PA (US)

(21) Appl. No.: 18/880,435

(22) PCT Filed: Aug. 22, 2024
(86) PCT No.: PCT/US24/43341

§ 371 Date: Dec. 31, 2024

Related U.S. Application Data
(60) Provisional application No. 63/534,501, filed on Aug. 24, 2023.

(51) Int. Cl. G06N 3/09

(52) U.S. Cl. CPC G06N 3/09

(57) ABSTRACT

Disclosed herein is a posture-invariant brain-computer interface (BCI), including a neural decoder that effectively provides correct motion intent signals even as the user changes posture, a method of training the neural decoder using a supervised learning method to predict intended effector motion from training data collected in multiple postures, and a system using the trained neural decoder to effect intended motions on an effector.

### RELATED APPLICATIONS

This application is a national filing under 35 U.S.C. § 371 claiming the benefit and priority to International Patent Application No. PCT/US24/43341, filed Aug. 22, 2024 entitled “TRAINING AND USE OF A POSTURE INVARIANT BRAIN-COMPUTER INTERFACE”, which claims the benefit of U.S. Provisional Patent Application No. 63/534,501 filed Aug. 24, 2023, the contents of which are incorporated herein in their entireties.

### GOVERNMENT INTEREST

This invention was made with the support of the United States government under contracts HD090125, HD071686, NS120579, and NS129584 awarded by the National Institutes of Health. The U.S. government has certain rights in the invention.

### DEFINITIONS

As used herein, the terms “brain-computer interface”, “BCI” and “neural decoder” are used interchangeably, although the brain computer interface typically consists of a neural decoder, with other components described herein.

### BACKGROUND OF THE INVENTION

Brain-Computer Interfaces (BCIs) are devices that infer a user's intent from neural activity, then use the inferred intentions to control a device, for example, a computer cursor, a robot arm, or some other type of external device (i.e., the effector). Often, the neural activity used to control a BCI is recorded from the primary motor cortex (M1). However, M1 contains a variety of signals besides those carrying intent information, potentially making it challenging to extract an intent signal uncorrupted by other signals. One such confounding signal in M1 is related to the posture or configuration of the subject's body while using the BCI. Changes in posture-related signals in M1 are expected during BCI control, as, for example, in the instance of a paralyzed user sitting upright or laying down. Furthermore, if a BCI succeeds in restoring natural limb motion, postural changes may become even more frequent. Changing posture causes a degradation in the performance of standard BCIs, requiring a time-consuming recalibration in each new posture. Thus, it would be desirable to create a BCI that can infer user intent regardless of body posture. Existing systems for understanding the effects of changing posture on neural activity during BCI use are limited, which makes the design of a posture-invariant BCI challenging.

To generate movement commands, the brain must combine information about the goal of the movement with information about the posture of the body. M1 is a key node for the convergence of these information streams and is the primary source of descending motor commands for volitional movement.

As an example of the interaction between goal and posture, consider that it is cognitively easy to reach in a given direction, regardless of the initial configuration, or posture, of the arm. However, altering the initial posture from which a movement is made necessitates complicated changes in the muscle activity required to make it happen. Populations of neurons in the brain rapidly and flexibly combine information about arm posture and movement goal to compute muscle commands in the moments before a movement is executed.

The primary motor cortex (M1) plays an important role in this process. It receives inputs about arm posture and movement goal from sensory and association areas and in turn provides the major source of descending projections to the spinal cord for the control of the arm and hand. Changing the initial posture from which a movement is made can cause complicated changes in the activity of individual M1 neurons.

### SUMMARY OF THE INVENTION

As shown in FIG. 1, posture and goal modulate distinct subspaces of neural activity. There exists a clear and simple organization of posture information in M1. First, neural population activity can be decomposed into posture-related and goal-related components that are separated into nearly orthogonal subspaces. Second, a single subspace can be used to decode arm posture across tasks, even though posture has very different implications for each task. Third, the overall geometry of neural population activity is highly similar across postures. Together, these results indicate the existence of a ‘posture subspace’ in M1 activity. That is, posture produces similar effects in neural population activity across a wide range of behaviors, and those effects are confined to a unique subspace. The organization of posture and goal information into separate subspaces likely facilitates the brain's ability to easily combine the two flexibly.

Disclosed herein is a posture-invariant brain-computer interface (BCI) that maintains high performance (i.e., discerning intent) even as the user changes posture. To calibrate the BCI (i.e., to learn the parameters of the model relating neural activity to a desired motion), a supervised learning method is used on training data collected in multiple postures. The method leverages intent-related structure in neural signals that is shared across postures and ignores posture-related structure, resulting in a BCI that works regardless of the posture of the subject. The training procedure requires collecting data in sample postures, then a BCI is produced that works across a wide range of postures without the need for recalibration.

The posture-invariant BCI exhibits improved performance over a standard BCI when human subjects, both paralyzed and able-bodied, change posture during BCI use. The primary advantage of the posture-invariant BCI is that it does not require recalibration for each new posture.

In some embodiments, techniques underlying calibration procedures for posture-invariant BCIs include collecting training data in multiple postures, and/or using supervised learning to predict desired effector motion (i.e., the “intent”) from one or more training datasets. In some embodiments, techniques underlying calibration procedures for posture-invariant BCIs include collecting training data in multiple postures. In each posture, subjects may attempt to make the same set of effector movements (e.g., as a proof-of-concept, point-to-point cursor movements in a variety of directions). In some embodiments, sufficient data is collected in each posture to appropriately calibrate a BCI.

### BRIEF DESCRIPTION OF THE DRAWINGS

FIG. 1 is a schematic diagram showing the orthogonal subspaces of posture-related and goal-related neural activity.

FIG. 2 is a schematic diagram showing the BCI task used to study the effects of posture and goal on neural activity.

FIG. 3A (top row) comprises graphs showing trial-averaged channel responses for the rightward movement goal for each arm posture. FIG. 3A (bottom row) comprises graphs showing trial-averaged channel responses for all movement goals when the arm was in the innermost (red) posture. FIG. 3B schematically shows that the two different dimensionality reduction approaches used for visualization (PCA and Targeted Dimensionality Reduction) produce different low-dimensional projections of the same high-dimensional neural data.

FIGS. 4(A,B) are graphs showing trial-averaged neural trajectories for all movement goals and postures for a subject in space identified using PCA and targeted dimensionality reduction, respectively.

FIGS. 5(A-C) are graphs showing trial-averaged neural trajectories for all movement goals and postures for a subject in space identified using targeted dimensionality reduction for different subjects and posture manipulations: using a 45 degree shoulder rotation(A); using 45 degree elbow and shoulder rotations (B); and using 90 degree shoulder abduction and 45 degree shoulder rotation (C).

FIGS. 6(A,B) are graphs showing the projections of the posture and goal components of neural activity into the posture and goal subspaces, illustrating that the subspaces representing posture and goal are nearly orthogonal.

FIG. 7A is a schematic showing the isometric force task. FIG. 7B is a graph showing the trial-averaged neural trajectories for all movement goals and postures for a subject in space identified by targeted dimensionality reduction FIG. 7C is a graph showing posture-related variance captured by posture and goal subspaces for the isometric force task.

FIG. 8A is a schematic showing the reaching task. FIG. 8B is a graph showing the trial-averaged neural trajectories for all movement goals and postures for a subject in space identified by targeted dimensionality reduction. FIG. 8C is a graph showing posture-related variance captured by posture and goal subspaces for the reaching task.

FIG. 9A is a schematic showing a multiple task paradigm. FIG. 9B comprises graphs showing projections of average neural activity for individual trials onto the posture subspace identified by linear discriminant analysis for different postures (upper left) the reaching task (upper right), the isometric force task (lower left) and a composite of the posture, reaching task and isometric force task (lower right). FIG. 9C is a graph showing a 5-fold cross-validated posture classification accuracy using LDA in 3 different scenarios, wherein, for each scenario, bar height and error bar are the mean and SEM of the cross-validated classification accuracies. FIG. 9D is a graph showing the neural activity can be separated by task, indicating that it is distinct across tasks.

FIG. 10A illustrates the procedure for measuring the difference, or error, between neural trajectories. Trajectories for the same movement goal, but different postures, are compared (left). The ‘error before shift’ is measured as the mean Euclidean distance between the two trajectories. The blue trajectory is mean-shifted to compensate for any offset between the two trajectories (center). The error after shift is measured as the mean Euclidean distance remaining between the two trajectories. (right). FIG. 10B is a graph showing hypotheses stating that trajectories may have either reshaped strongly across postures (top), in which case the error before and after shifting would be similar, producing points near the diagonal, or trajectories may have displayed little-to-no reshaping (bottom), producing points for which the normalized error after shift was nearly 1. FIG. 10C are graphs showing error after shift vs. error before shift for all subjects and tasks. Each point is one comparison (e.g., posture 1 goal 1 vs. posture 2 goal 1). The “X” markers indicate the mean for each subject. Errors before and after shifting were normalized by the error between trajectories subsampled from the same experimental condition.

FIG. 11 is a schematic diagram of an exemplary system implementing a trained brain-computer interface in accordance with this invention.

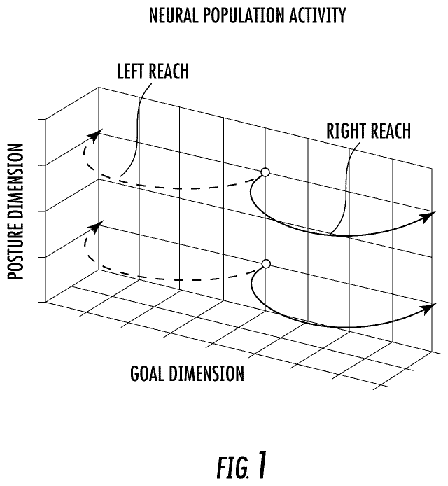

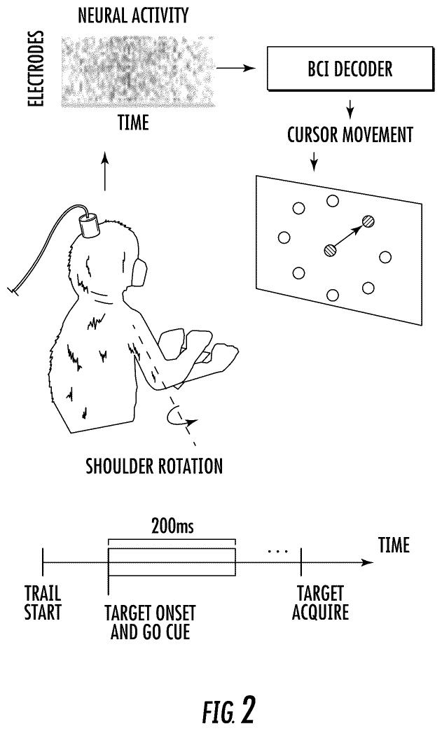

### DETAILED DESCRIPTION

Disclosed herein is a method for training and calibrating a posture-invariant BCI. Because of the structure in neural signals discussed above, the method results in a BCI that works across postures. This is because appropriate supervised learning models concentrate on the structure in intent-related signals that is shared across postures while suppressing the irrelevant posture signals.

It must first be understood how arm posture and the movement goal (intent) affect neural population activity in the primary motor cortex (M1) during a BCI task. In one embodiment, an exemplary BCI task consisted of subjects moving a cursor on a computer screen from a variety of different postures. These exercises provide a powerful tool for determining the effects of posture on neural activity. This is because, during the use of a BCI, arm posture is static while M1 neurons are actively performing a movement goal (e.g., controlling the computer cursor). This minimizes the confounding influence of time-varying proprioceptive signals that are present in M1 activity during tasks involving overt arm movements.

A series of experiments, described below, were conducted to discover the relationship between posture signals and intent signal in a neural population. It was discovered that posture signals modulate dimensions of neural activity that are different from those modulated by intent signals. Further, posture signals are consistent across a variety of tasks and intent signals can be preserved across postures.

In the exemplary BCI task, subjects volitionally modulated M1 activity to move a computer cursor from the center of a virtual reality workspace to one of eight indicated radially-arranged movement goals, as shown in FIG. 2. The posture of the arm contralateral to the recording array was changed in blocks, and a new decoder was calibrated for each new posture. The subjects neither made arm movements nor exerted large forces with their hands during BCI control. The observed postural effects were still present when the same decoder was used across postures.

Neural activity was examined in a 200 ms window beginning at the go cue, which coincided with the appearance of the peripheral goal that the subject acquired during the task. This window was chosen to be long enough to include strong goal-related effects on neural activity while being short enough to exclude neural activity related to corrective cursor movements.

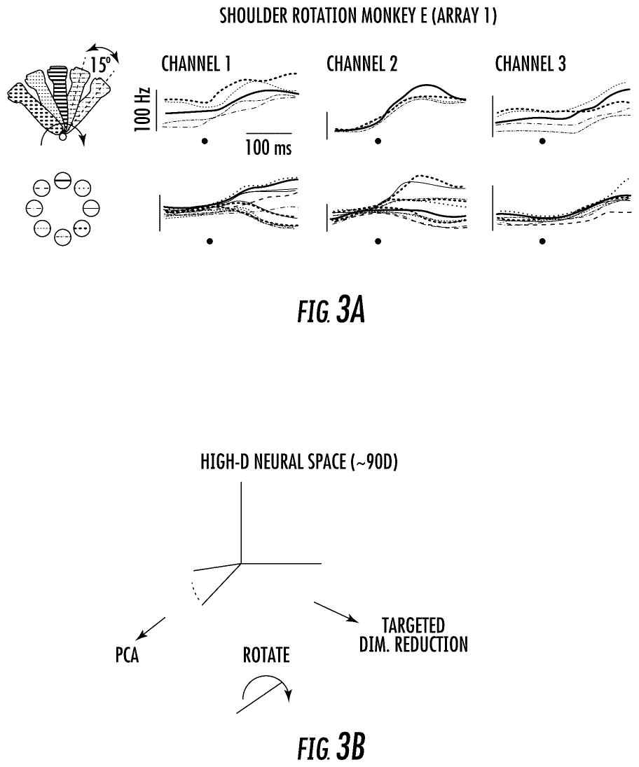

The responses of individual neural channels during the task are shown in FIG. 3A. Most channels exhibited mixed tuning to posture and goal (channel 1), while some were tuned exclusively to goal (channel 2) or posture (channel 3). Postural tuning was present both before and during cursor movement, as can be seen in the responses for channels 1 and 3. Many channels also exhibited changes in goal tuning across postures.

To study the effects of posture and goal at the neural population level, both principal component analysis (PCA) and a targeted dimensionality reduction approach were applied to the single unit recordings. As shown in FIG. 3B, two different low-dimensional projections of the same high-dimensional neural activity were produced, one using PCA and one using targeted dimensionality reduction. The neural dimensions identified by the targeted dimensionality reduction approach are related to the dimensions identified by PCA by a rotation.

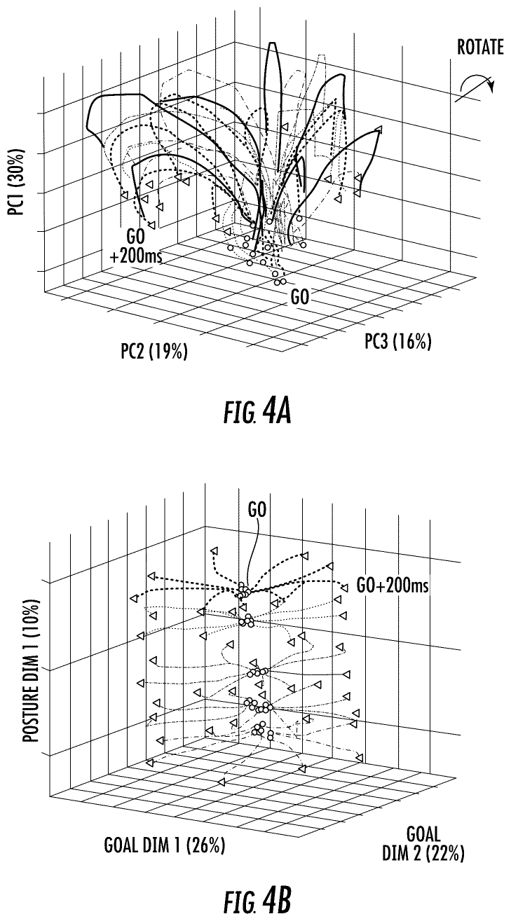

As shown in FIG. 4A, when data from all task conditions were visualized in the top principle components, the neural time courses, or neural trajectories, displayed little discernible organization. Analysis of trajectories for the same goal, however, revealed that trajectories were similar across postures, with small offsets in some PCs. This suggested that there are separable effects of posture and goal in neural population activity.

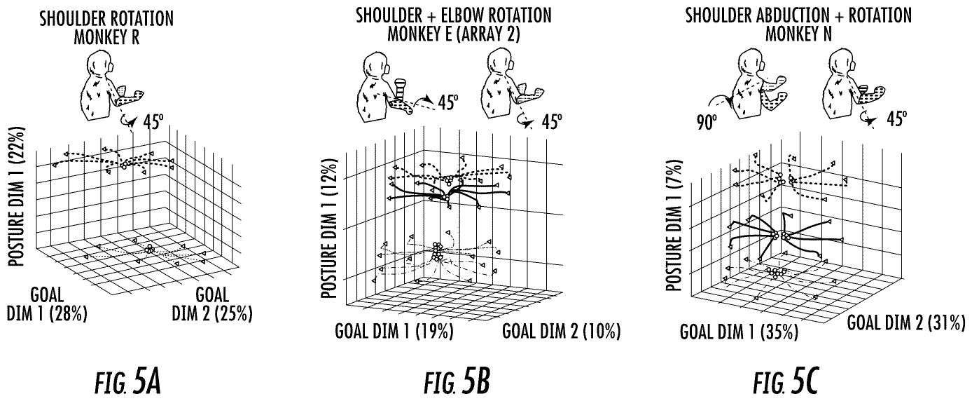

To better isolate the effects of posture and goal, neural activity was projected onto the dimensions identified by the targeted dimensionality reduction approach, as shown in FIG. 4B. When projected into these dimensions, neural trajectories exhibited clear spatial structure, with separable effects of posture and goal. Neural trajectories are shown to each of the 8 movement goals, for each of the postures. Trajectories to different movement goals within a posture emanated outward in different directions along goal dimensions. When the arm's posture was changed, the structure of these neural trajectories was conserved, with all trajectories offset along posture dimensions. A similar structure was observed using other algorithms for identifying the posture and goal dimensions. The results from a first subject are shown in FIG. 5A. The experiments were repeated with additional joint rotations including elbow rotation (FIG. 5B) and shoulder abduction (FIG. 5C), and in each case, changing the posture of the arm shifted neural trajectories along posture dimensions, while the shapes of the neural trajectories were conserved.

These results indicate that posture and goal have separable effects on neural population activity during BCI control: changes in posture offset neural trajectories along posture dimensions, while changes in goal alter the direction in which trajectories emanate along goal dimensions.

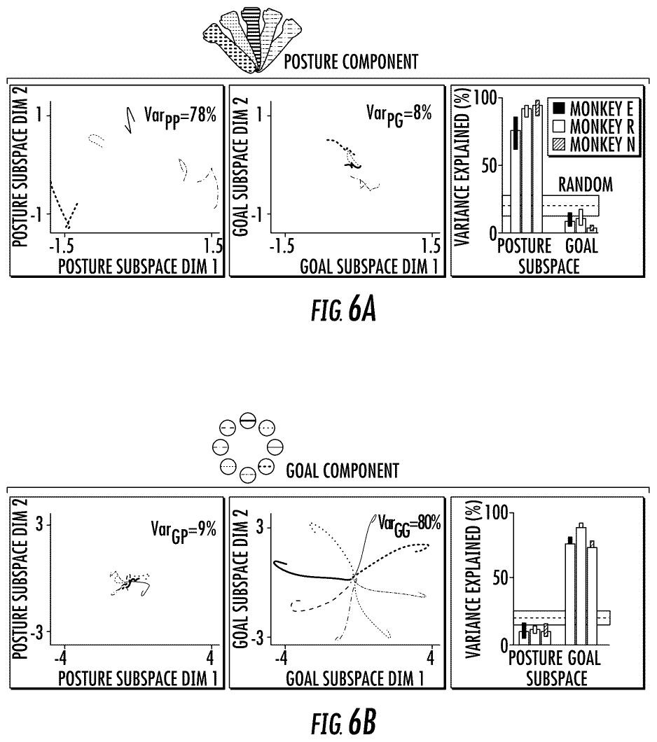

Distinct subspaces of neural activity are useful for isolating signals The separation of posture and goal information into substantially orthogonal subspaces enables the brain to combine them flexibly. At one extreme, these dimensions could be orthogonal, as shown in FIG. 6A. At the other, they could be aligned, as shown in FIG. 6B.

To quantify the alignment of the posture and goal dimensions, a cross-projection alignment test was performed. A marginalization approach was first used to decompose neural population activity into posture and goal components. PCA was then used to identify dimensions which captured variance in the posture and goal components separately, which is referred to herein as the “posture subspace” and “goal subspace”, respectively. The posture component was then projected into each subspace. If the subspaces were aligned, they would capture the same amount of posture-related variance. If they were orthogonal, then the goal subspace would capture little posture-related variance. This procedure was repeated with the goal component.

The posture subspace was found to capture little goal variance. For example, for the session shown in FIGS. 6(A,B), a 2-dimensional posture subspace captured 78% of the posture variance, while a 2-dimensional goal subspace captured only 9%. This difference could be seen for all subjects, as shown in the graph in FIG. 6A. Across subjects, the goal subspace typically captured less posture variance than randomly drawn subspaces, indicating that posture and goal subspaces were highly misaligned. For sessions in which multiple joints were tested, different joints modulated different posture dimensions, all of which were highly misaligned with goal dimensions.

Similarly, the goal subspace captured little posture variance. In the session shown, the 2-D posture subspace captures 9% of goal variance, while the 2-D goal subspace captures 80% of it. As shown in the graph in FIG. 6B, this effect was observed for all subjects.

The BCI paradigm enabled the isolation of the effects of posture and goal on neural activity by removing many of the confounding factors present during overt movement, such as time-varying sensory feedback and changes in movement commands across postures. This revealed that, during BCI control, posture and goal modulate distinct subspaces of M1 neural population activity.

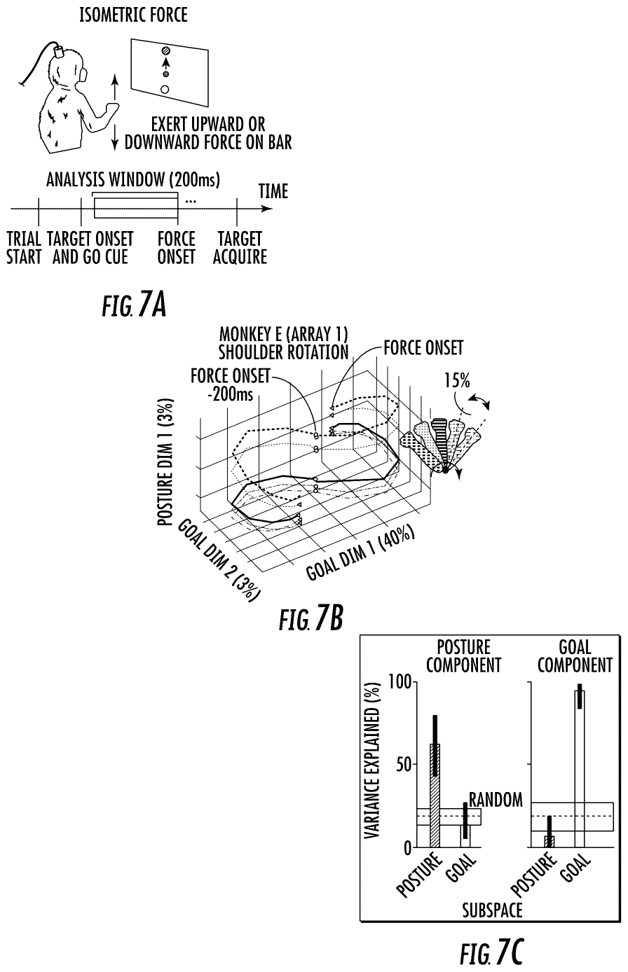

Because neural activity during tasks involving muscular contraction was not part of the BCI control, these activities may be very different from those observed during BCI control. As such, no conclusion can be made as to whether a similar organization of neural activity exists during ‘overt’ isometric force and reaching tasks, which require muscular contraction. Therefore, data from the isometric force task was also collected. In this task, as shown in FIG. 7A, muscle activation is required for task success, but limb movement is restricted, mitigating the influence of time-varying proprioceptive feedback. The subject exerted upward or downward forces on a handle attached to a force transducer to move a computer cursor to a movement goal displayed on the screen. Arm posture was changed by rotating the forearm about the shoulder. Neural activity was analyzed in the 200 ms before force onset to include strong goal effects on neural activity while excluding as much as possible reafferent proprioceptive feedback from the contracting muscles.

When neural population activity was examined in the isometric task, neural trajectories for each movement goal showed conserved structure, separated along posture dimensions, as shown in FIG. 7B. The subspace alignment analysis was repeated and again it was shown that posture and goal subspaces were nearly orthogonal, as show in the graph in FIG. 7C.

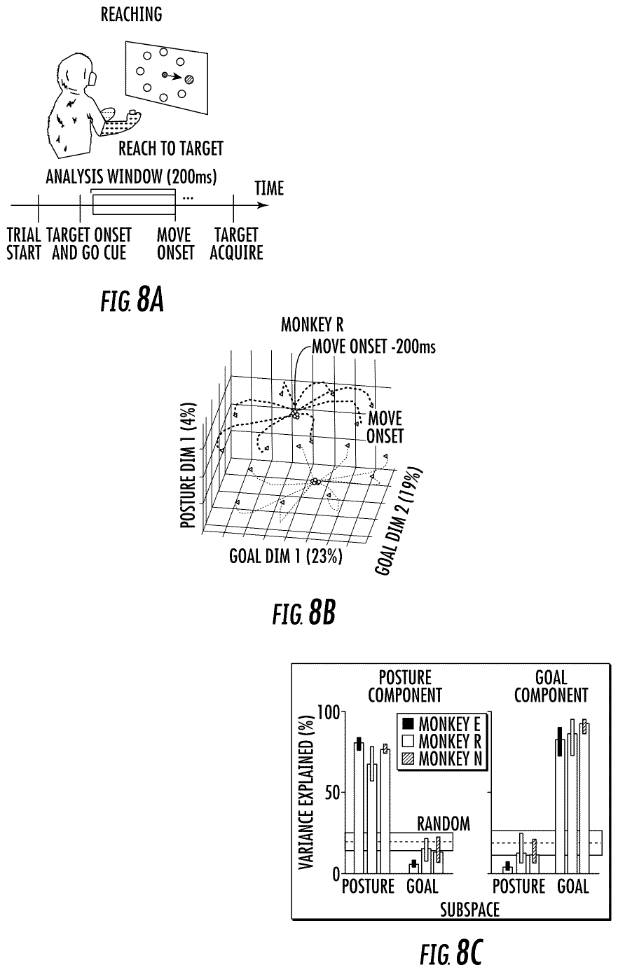

This same organization of posture and goal effects was found to be present during a center-out reaching task performed from different initial arm postures. Similar tasks produce substantial changes in muscle activation and the activity of individual neurons across postures. In this task, subjects made reaches to one of eight radially-arranged targets. The initial posture of the arm was changed in blocks by changing the initial location of the hand in the frontoparallel plane, as shown in FIG. 8A. Reaches were matched in direction and length from each initial hand position. Visual feedback was matched across postures so that the instructed initial hand position always corresponded to the center of the screen. Neural activity was analyzed in the 200 ms preceding movement onset to exclude both movement and reafferent feedback.

Even during reaching, a clear organization of posture and goal information in neural population activity was present, as shown in FIG. 8B. Posture and goal subspaces were nearly orthogonal, as shown in the graph in FIG. 8C. During later periods of the task, when the arm was moving, neural activity in the posture dimensions did not closely track arm position. This may be due to the fact that muscle lengths change in complicated ways during reaching, and that the posture dimensions identified here capture changes in neural activity associated with the specific changes in muscle lengths between initial static postures. Within each of the BCI, isometric force, and reaching tasks, neural trajectories seemed to have a similar overall geometry for a given movement goal.

Together, these results suggest that the separation of posture and goal information into distinct subspaces is an organizing principle of motor cortex, easiest to see in the BCI case, but also present during behaviors that engage the muscles and move the arm.

Across a wide variety of tasks, posture and goal information were separated into distinct neural subspaces. Is there a single ‘posture subspace’ in M1 which indicates the arm's posture, regardless of the task being performed? Posture need not be encoded in this way in M1. This is because a change in arm posture can necessitate task-specific changes in muscle output. Given this consideration, it is also reasonable to expect that posture-related signals in neural activity are task-specific.

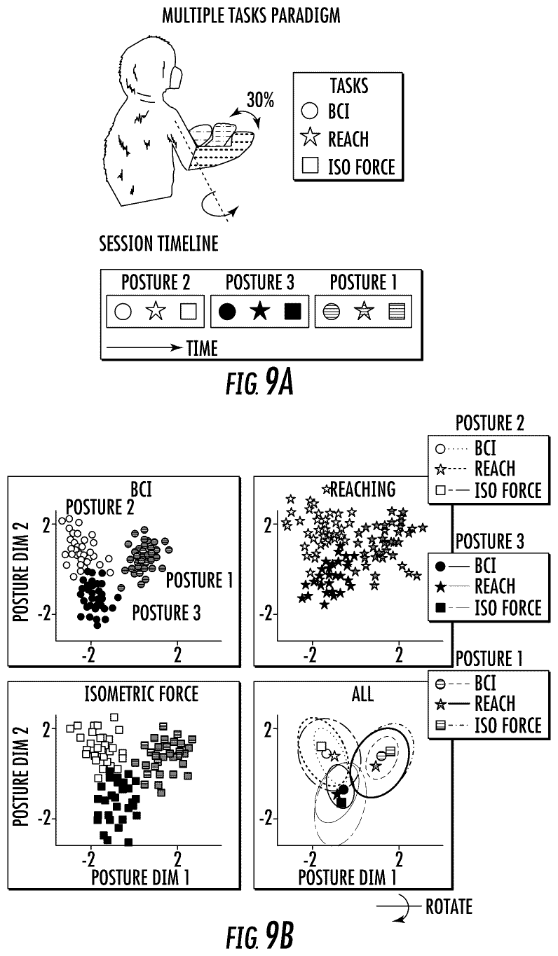

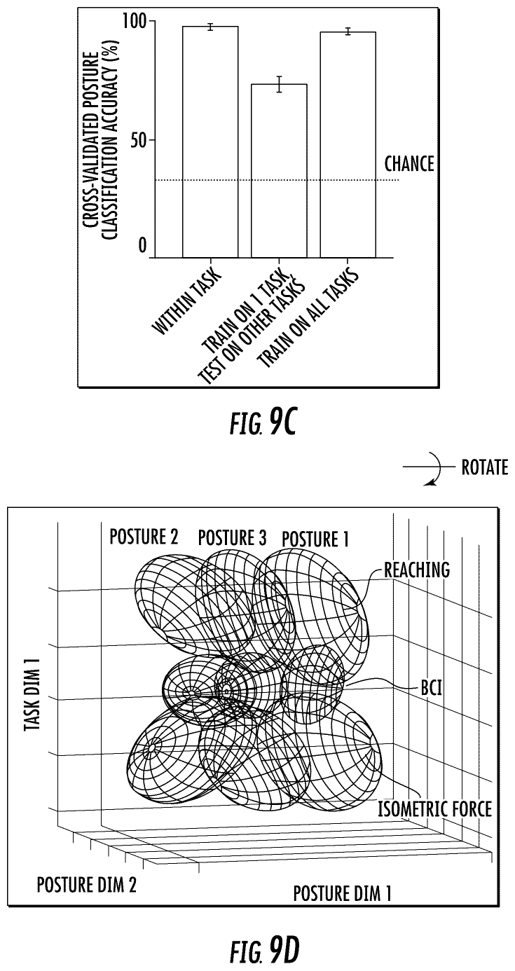

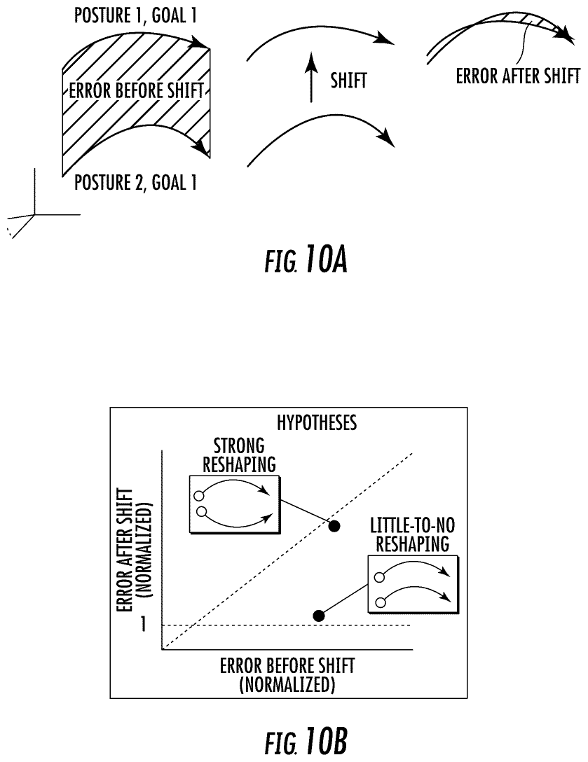

To disambiguate between these possibilities, one subject completed a “multiple tasks” paradigm, in which all three of the tasks were performed within a single experimental session, as shown in FIG. 9A. To determine if it is possible to identify a single subspace from which arm posture can be read out across tasks, neural activity was first averaged across the analysis window previously indicated for each task, resulting in one observation (i.e., a spike count vector) per trial. Linear discriminant analysis (LDA) was then used to identify a subspace that separated data from all tasks by posture. Projections of each task's data into the subspace identified by LDA were qualitatively similar, as shown in FIG. 9B, suggesting that it may be possible to use this subspace to classify posture across tasks.

Therefore, LDA was used to classify posture from neural activity. To establish a baseline for classifier performance, classifiers were trained on each task separately, and the cross-validated prediction accuracies were nearly 100%, as shown in the graph in FIG. 9C. When a single decoder was used for all tasks, there was hardly any degradation in performance. An even stronger test of the consistency of the posture subspace across tasks is to train the decoder on one task and test on others. The classifiers still performed well above chance levels, with a moderate decrement in performance, although this decrement could be due to overfitting.

A trivial explanation for the ability to decode posture using a single subspace across tasks is that neural activity is very similar across tasks. However, other neural dimensions were identified that strongly separated neural activity across tasks, indicating that neural activity in each task was starkly different, as shown in FIG. 9D. This suggests that the posture subspace is consistent across tasks, even though overall neural activity is not. Together, these analyses support the existence of a posture subspace in M1's population activity.

From the above discussion, posture and goal information are separated in M1 neural population activity across a variety of motor tasks and the posture subspace is consistent across tasks. A surprising feature of neural activity that contributed to this organization is that the overall geometry of neural trajectories was similar across postures. This similarity could indicate that goal-related patterns of neural activity are reused across postures when generating movement commands. Next, the extent to which trajectories changed shape across postures was quantified.

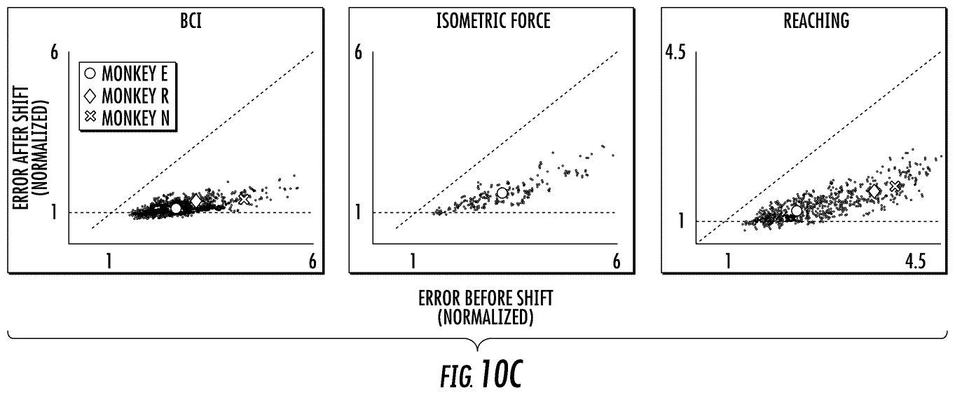

The mean Euclidean distance between them was measured before and after removing the offset, as shown in FIG. 10A. This distance is referred to herein as the “error” between trajectories. If trajectories did not reshape at all, then removing any offset between them should result in a low “error after shift”, as shown in FIG. 10B. Alternatively, if trajectories reshape strongly, then removing the offset will not completely remove the error between them, resulting in a higher “error after shift”. To provide a scale for the errors, each error was normalized by the error between trajectories subsampled from within the same experimental condition. Therefore, an “error after shift” of 1 indicates that, once the offset between them has been removed, trajectories from across postures are as similar in shape as trajectories from within a condition.

Across all tasks, removing the offset between trajectories accounted for nearly all of the error between them, as shown in FIG. 10C. Thus, even during overt movements, the dominant aspects of the neural population response are conserved across postures.

The method disclosed herein teaches the calibration of a BCI using a supervised learning method to predict intended effector motion from training data collected in multiple postures. The method includes, first, collecting training data in multiple postures. In each posture, the subject attempts to make the same set of effector movements (e.g., point-to-point cursor movements in a variety of directions). Sufficient data must be collected in each posture to appropriately calibrate the BCI. Second, the method includes the use of supervised learning to predict the desired effector motion (i.e., ‘intent’) from the training dataset.

Because of the structure in neural signals, this method results in BCIs that successfully predict intent across postures. This may be because appropriate supervised learning algorithms may concentrate on structures in intent-related signals that may be shared across postures, while suppressing irrelevant posture signals.

The BCI may be implemented in a number of different ways. For example, a machine learning model or neural network may be trained using a supervised training method (using any loss functions, for example, mean squared error loss or cross entropy loss) to discern intent from raw neural data. Any machine learning model or neural network architecture may be used for this purpose. However, because the posture and intent information occupy different subspaces of the neural activity, linear decoding methods may also be successfully used to separate the intent information from the posture information. In various embodiments hereon, any linear decoding method may be used, however, the invention is explained in the context of the use of a Kalman filter for linear decoding.

The steps of the method may be implemented using a variety of approaches. In a non-limiting example, cursor velocity may be the predicted variable, and the supervised learning includes two steps: First, the dimensionality of the training data is reduced using linear discriminant analysis, which helps to exclude posture signals and concentrate on intent signals. Next, the low-dimensional data is used to predict cursor velocity at each timestep using a Kalman filter.

Neural data is recorded from multi-electrode arrays and pre-processed using standard techniques. The pre-processing may comprise, in one embodiment, one or more of amplification of raw voltage signals (e.g., 1,000-32,000 times), bandpass filtering (e.g., 250-8,000 Hz), digitizing, and thresholding (e.g., at 3×RMS for each channel). Threshold crossings are then binned (e.g., 50 ms, non-overlapping bins).

To collect the training dataset used for calibrating the posture-invariant BCI, the following procedure is repeated in each of several postures. First, a closed-loop calibration procedure (e.g., 160 trials) is used to calibrate a “posture-specific” BCI for the current posture. Then, that BCI is used to collect new trials of training data (e.g., 160 trials). Finally, the trials of training data from each posture are combined to form the final training dataset used to calibrate a single posture-invariant BCI.

Center-out task: A standard 8-target center-out task, as described above, is used for both the closed-loop calibration procedure and the training data collection. On each trial of this task, the subject moves a cursor from a central target to one of 8 radially-arranged targets. Target directions are pseudorandomized across trials.

Closed-loop calibration procedure: A closed-loop calibration procedure is used for each posture-specific BCI. In one embodiment subjects complete 5, 32-trial blocks of the center-out task. In each block, subjects gain progressively more control over the BCI. The first block is an “observation block” during which neural activity is recorded while the user observes a computer cursor moving from the center to a peripheral target. The data from this block is used to calibrate an initial posture-specific BCI. In the 2nd block, the initial BCI is used to control the cursor, but the cursor's movement is restricted so that it moves in a straight line towards the target (i.e., any cursor movement perpendicular to the target is scaled by a factor of 0). Before each subsequent block, a new BCI is calibrated using all closed-loop trials (i.e., trials from the 2nd block onward), and the factor by which cursor motion perpendicular to the target is scaled is increased. In one embodiment, the scaling factors for the 2nd-5th blocks are [0,0.25,0.5,1]. The final posture-specific BCI is calibrated using data from the 2nd-5th blocks and has a scaling factor of 1.

Posture-invariant intent estimation: After the training dataset is collected, linear discriminant analysis (LDA) is used to identify a low-dimensional projection of the training data from which intended velocity can be easily inferred, regardless of posture. LDA identifies a projection that simultaneously maximizes separation between classes and minimizes variance within each class. Therefore, labelling the training data by target direction without regard to posture allows LDA to capitalize on shared intent-related structure across postures, while ignoring posture-related variability. This results in a projection in which data from each target direction are well-separated, and within each target cluster, data from each posture are squashed together, as explained below.

First, the training data is z-scored and organized into a matrix N∈ [symbol] ^{D×o}, where D is the number of recorded neural channels and o is the number of observations (e.g., time steps). Each observation is 1 bin of neural spike counts. (Note: if N is singular or nearly singular, its dimensionality must first be reduced with PCA or by applying a regularized version of LDA). A class label is assigned to each observation, where the label corresponds to the target for the trial on which the observation was recorded.

Next, the mean of each class, mᵢ∈ [symbol] ^{D×1} where i=1, 2, . . . , c is computed as

$${m}_{i} = \frac{1}{{n}_{i}} {\sum }_{{N}_{j} ∈ {C}_{i}} {N}_{j}$$

where:
Nⱼ∈ [symbol] ^{D×1} is a single observation of neural data; and
nᵢ is the number of observations for the i^{th} class, C.

The “between-class scatter”, S_{b}∈ [symbol] ^{D×D} is then computed as:

$${S}_{b} = {\sum }_{i = 1}^{c} {n}_{i} ( {m}_{i} - m ) {( {m}_{i} - m )}^{T}$$

where c is the number of classes; and

$$m = \frac{1}{c} {\sum }_{i = 1}^{c} {m}_{i}$$

is the mean of the class means.

Similarly, the “within-class scatter”, S_{w}∈ [symbol] ^{D×D} is computed as:

$${S}_{w} = {\sum }_{i = 1}^{c} {\sum }_{{N}_{j} ∈ {C}_{i}} ( {N}_{j} - {m}_{i} ) {( {N}_{j} - {m}_{i} )}^{T}$$

The dimensions v∈ [symbol] ^{D×1} which maximize the ratio of between-class to within-class scatter are then discovered:

$${max}_{v : ‖ v ‖ = 1} \frac{{v}^{T} {S}_{b} v}{{v}^{T} {S}_{w} v}$$

by solving the generalized eigenvector problem:

$${S}_{b} v = λ {S}_{w} v$$

where:
λ is a diagonal matrix of eigenvalues.

There are at most c−1 eigenvectors, and only the first d of these will need to be kept (i.e., the number needed to accurately decode intended velocity). These are collected into a matrix V∈ [symbol] ^{D×d}.

Now, each observation of neural activity may be projected into this low dimensional space for intent estimation, producing a new, lower-dimensional set of observations, X∈ [symbol] ^{d×o}

$$X = {V}^{T} N$$

This low-dimensional projection can then be used to calibrate BCIs using traditional techniques, such as Kalman filtering, optimal linear estimation, neural networks, or similar. The Kalman filter approach is outlined below for illustrative purposes.

Integration with Kalman Filter: The data is then used to estimate parameters for a Kalman filter which predicts 2-d cursor velocity at each timestep, as explained below. The intended velocity at each timestep in the training dataset is taken to be a vector pointing from the center of the workspace to the current target with a constant magnitude.

Let zₜ∈ [symbol] ^{M×1} be the cursor state (for 2-d velocity, M=2) at time t, xₜ∈ [symbol] ^{d×1} be the neural state at time t (i.e., one column from the matrix X above), where d is number of number of dimensions remaining after projection into the subspace identified with LDA.

The Kalman filter is defined by (1) the state model:

$${z}_{t} \vert {z}_{t - 1} ∼ N ( {Az}_{t - 1} , Q )$$

$${z}_{1} ∼ V ( π , V )$$

where:
π∈ [symbol] ^{M×1} and V∈ [symbol] ^{M×M} are the mean and covariance of the initial cursor state, respectively.

The Kalman filter is further defined by (2) the observation model:

$${z}_{t} \vert {z}_{t} ∼ N ( {Cz}_{t} , R )$$

The model is fit using maximum-likelihood estimation:

$$A = ( {\sum }_{t = 2}^{T} {z}_{t} {z}_{t - 1}^{T} ) {( {\sum }_{t = 2}^{T} {z}_{t - 1} {z}_{t - 1}^{T} )}^{- 1}$$

$$Q = \frac{1}{T - 1} {\sum }_{t = 2}^{T} ( {z}_{t} - {Az}_{t - 1} ) {( {z}_{t} - {Az}_{t - 1} )}^{T}$$

$$C = ( {\sum }_{t = 1}^{T} {x}_{t} {z}_{t}^{T} ) {( {\sum }_{t = 1}^{T} {z}_{t} {z}_{t}^{T} )}^{- 1}$$

$$R = \frac{1}{T} {\sum }_{t = 1}^{T} ( {x}_{t} - {Cz}_{t} ) {( {x}_{t} - {Cz}_{t} )}^{T}$$

where:
T is the total number of time steps in the training data.

A and Q are the parameters of the state model of the Kalman filter. They describe how the previous cursor state relates to the next one. The state model says that, given the previous cursor state, zₜ₋₁, the current cursor state, zₜ, is normally distributed with a mean of A times zₜ₋₁ and a covariance of Q. This means that the current cursor state is linearly related to the previous one, but this relationship is noisy. Q is sometimes called the “process noise”.

C and R are the parameters of the observation model of the Kalman filter. They describe how the current cursor position relates to the current sample of neural activity. The observation model says that, given the current cursor state, zₜ, the current sample of neural activity, xₜ, is normally distributed with a mean of C times zₜ and a covariance of R. This means that neural activity is linearly related to the cursor position, but this relationship is noisy. R is sometimes called the “observation noise”.

To predict cursor states, first define:

$${μ}_{t}^{τ} = E [ {z}_{t} \vert {\{ x \}}_{1}^{τ} ]$$

$${\sum }_{t}^{τ} = cov [ {z}_{t} \vert {\{ x \}}_{1}^{τ} ]$$

where:

$${μ}_{t}^{τ} and {\sum }_{t}^{τ}$$

are the posterior mean and covariance of zₜ given neural activity from timestep 1 to τ, respectively.

Then, one-step predictions of the cursor state

$${μ}_{t}^{t - 1}$$

and its covariance

$${\sum }_{t}^{t - 1}$$

are made as:

$${μ}_{t}^{t - 1} = {μ}_{t - 1}^{t - 1}$$

$${\sum }_{t}^{t - 1} = A {\sum }_{t - 1}^{t - 1} {A}^{T} + Q$$

and updated with measurements to form estimates

$${μ}_{t}^{t} and {\sum }_{t}^{t}$$

as:

$${K}_{t} = {\sum }_{t}^{t - 1} {{C}^{T} ( C {\sum }_{t}^{t - 1} {C}^{T} + R )}^{- 1}$$

$${μ}_{t}^{t} = {μ}_{t}^{t - 1} + {K}_{t} ( {x}_{t} - C {μ}_{t}^{t - 1} )$$

$${\sum }_{t}^{t} = {\sum }_{t}^{t - 1} - {K}_{t} C {\sum }_{t}^{t - 1}$$

where:
Kₜ is the Kalman gain.

In the context of the exemplary embodiment, wherein the BCI controls a computer cursor, the output of the Kalman filter is an estimation of cursor state (e.g., the position and velocity of the cursor at the current time). In the equations above, and in the context of the example application where in desired motion is movement of a cursor on a screen.

$${μ}_{t}^{t}$$

is the cursor state and xₜ is a sample of neural activity. The form of the equations for the Kalman filter do not change, regardless of the system they are used to describe. However, the terms in the equations represent different variables for different systems. For example, the state of a computer cursor may be defined to consist of the 2-D (x and y) position and 2-D (x and y) velocity of the cursor. In such a case,

$${μ}_{t}^{t}$$

would be 4-dimensional, and if the variable values were positionₓ=0.2 meters, position_{y}=0.1 meters, velocityₓ=0.5 meters/second and velocity_{y}=0.4 meters/second, then

$${μ}_{t}^{t} = [ 0.2 , 0.1 , 0.5 , 0.4 ] .$$

When the system is a robotic hand, the system state may be defined to be the 3-D position and velocity of each of the 5 fingertips. In this case, a 30-dimensional system state (3 position dimensions and 3 velocity dimensions for each of the 5 fingers) would be represented by

$${μ}_{t}^{t} .$$

At a given moment in time, for example

$${μ}_{t}^{t} = [ 100 , 2.3 , 4 , … , 5.6 , 0.1 ]$$

(ellipses to indicate that

$${μ}_{t}^{t}$$

is 30 elements long). As would be realized, the meaning of

$${μ}_{t}^{t}$$

is highly dependent on the specific type of effector being controlled.

The algorithm is initialized with

$${μ}_{1}^{0} = π and {\sum }_{1}^{0} = V .$$

The procedure requires collecting training data in several postures, which may be time-consuming. However, if many postural changes are likely to occur within a day of BCI use, this procedure provides a marked improvement over repeated recalibrations in each new posture.

The BCI may be considered “fully calibrated” when it has been trained on enough data that its performance exceeds some threshold. For example, training data may be iteratively collected, and performance measured until the BCI user can acquire 8 radially-arranged targets in <8 seconds. In practice, the BCI may be considered fully calibrated once that much data has been collected and used to fit the BCI model, without a need to measure performance. Typically, BCIs are only recalibrated when necessary (perhaps once every few hours). However, in some approaches the BCI may be constantly updated during use. It should also be realized that a BCI may need to be separately trained for each individual subject, as the neural patterns of each subject vary for the same intent/posture.

While the invention has been explained in terms of a linear decoding method, it is also possible to determine intent signals from neural data using a trained machine learning model, such as a neural network. The neural data may be preprocessed and dimensionally reduced before being used as training/testing data for a neural network, but it does not need to be. Neural networks, when designed appropriately, have the capacity to effectively reduce the dimensionality of input data, so the step of dimensionality reduction may be unnecessary. As for preprocessing, the neural data must be digitized prior to use with a neural network (including the amplification and bandpass filtering steps), but this is the only constraint. After digitization, the data could be fed directly into the network or preprocessed in a variety of ways first, including, but not limited to, (1) thresholding and binning, or (2) being used to compute power in various frequency bands.

As would be realized, the invention is explained in terms of a model that relates neural activity to cursor movements on a screen. However, the invention may also be applied to affect other physical manifestations, for example, using a model relating neural activity to movement of a robotic hand to affect physical movement of the robotic hand.

The neural data may be collected by any known means, for example, by the placement of one or more invasive neural probes in or near the M1 area. It may also be possible to obtain the neural data non-invasively using, for example, EEG.

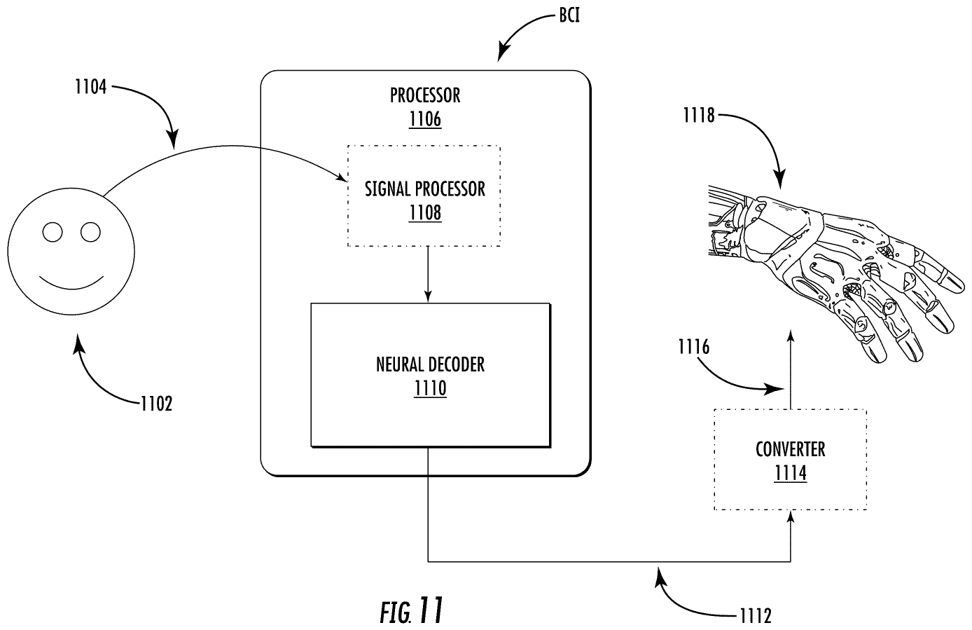

An exemplary system employing a trained BCI in accordance with this invention is shown in FIG. 11. Raw neural data 1104 is collected from subject 1102. Neural data 1104 is collected using one or more neural probes (not shown). The neural data 1104 may optionally be preprocessed, using one or more of the pre-processing steps previously described, by signal processor 1108. Signal processor 1108 may consist of circuitry or a software-implemented preprocessor running on processor 1106, or a combination of both. Processor 1106 may also execute the software implementing the trained neural decoder 1110, which outputs an estimation of the desired state 1112 of the effector 1118 reflected in the intent portion of the neural data 1104. The data representing the desired state 1112 may be optionally require conversion to one or more control signals 1116 by converter 1114 to effectively control effector 1118 to carry out the intended motions to reach the desired state. As would be realized, the implementation of converter 1114 is highly dependent on the type and specific implementation of effector 1118, and may be implemented by circuitry, software or a combination of both. Effector 1118 is shown as a robotic hand, but the invention is not meant to be limited thereby. Any effector 1118 may be controlled. Note that the BCI includes processor 1106, signal processor 1108, neural decoder 1110 and the neural probes (not shown), as well as any other hardware or software needed to determine the intent signals (e.g., user interface, etc.).

As would be realized by those of skill in the art, the specific examples discussed herein have been provided only as exemplary embodiments and the invention is not meant to be limited thereby. For example, the effector may be any controllable device, for example, a robotic hand. Modifications and variations are intended to be within the scope of the invention, which is given by the following claims:

1. A method of training a neural decoder comprising:
obtaining a training dataset comprising neural data collected from one or more subjects, the subjects performing various intent-driven activities from a plurality of initial postures; and
using a supervised learning method on the training dataset to learn parameters of the neural decoder such that the neural decoder predicts an intent of a subject, independent of the posture of the subject.

2. The method of claim 1 further comprising:
reducing the dimensionality of the training dataset.

3. The method of claim 1 wherein the neural decoder separates intent information from posture information, given an input of neural data from the subject.

4. The method of claim 1 further comprising:
preprocessing the neural data.

5. The method of claim 4 wherein preprocessing the data comprises:
amplifying raw voltage signals obtained from one or more data collection points;
bandpass filtering the signals;
digitizing the signals; and
thresholding the signals.

6. The method of claim 5 wherein the thresholded signals are binned in non-overlapping bins.

7. The method of claim 1 wherein obtaining a training dataset comprises:
performing a closed-loop calibration procedure to calibrate one or more posture-specific neural decoders;
using each neural decoder to collect new trials of training data as one or more subjects perform the various intent-driven activities; and
combining the new trials to form the training dataset.

8. The method of claim 7, wherein the close-loop calibration procedure comprises:
recording neural data from one or more subjects over multiple blocks of a task;
wherein a first block is an observational block during which neural activity is recorded while the user observes an intended motion to form an initial neural decoder;
wherein in a 2nd block, the intended motion controlled by the initial neural decoder;
wherein, in each subsequent block, the subjects are allowed increased control of the intended motion; and
wherein before each subsequent block, a new neural decoder is calibrated using all closed-loop trials.

9. The method of claim 1 wherein the neural decoder is a Kalman filter.

10. The method of claim 1 wherein the neural decoder is a trained deep neural network.

11. The method of claim 6 wherein the binned data is z-scored and organized into a D×o matrix, wherein D is the number of recorded neural channels and o is the number of observations.

12. The method of claim 2 wherein the dimensionality of the training dataset is reduced using principal component analysis.

13. The method of claim 2 wherein the dimensionality of the training dataset is reduced using a linear discriminant analysis.

14. The method of claim 2 wherein the low dimensionality data is used to estimate parameters of state and observational models of a Kalman filter.

15. A system comprising:
one or more neural probes for collecting neural data from a specific portion of a brain of a subject;
a processor;
software, implementing a neural decoder; and
an effector, for carrying out intended motions of the subject reflected in the neural data;
wherein the neural decoder has been trained to separate neural data indicating the intended motions from neural data indicating posture of the subject.

16. The system of claim 15 wherein the neural decoder separates intent information from posture information by reducing the neural data to low-dimensional data and applying a Kalman filter to the low-dimensional data.

17. The system of claim 15 wherein the neural decoder is a machine learning model trained to separate intent information from posture information.

18. The system of claim 15 wherein the neural data is preprocessed before being input to the neural decoder.

19. The system of claim 18 wherein preprocessing the neural data comprises:
amplifying raw voltage signals obtained from one or more of the neural probes;
bandpass filtering the raw voltage signals;
digitizing the signals; and
thresholding the signals.

20. The system of claim 15 wherein the effector is a robotic hand.

21. The system of claim 15 wherein the neural data is collected from the M1 region of the brain of the subject.
